(19) United States
(12) Patent Application Publication
(10) Pub. No.: US 2022/0323634 A1
(43) Pub. Date: Oct. 13, 2022

(54) FABRIC COMPOSITIONS COMPRISING ATTACHED ZEOLITE AND/OR A ZEOLITE/PECTIN COMPLEX

(71) Applicants: The United States of America, as represented by the Secretary of Agriculture, Washington, DC (US); H&H Medical Corporation, Williamsburg, VA (US); Virginia Commonwealth University, Richmond, VA (US)

(72) Inventors: Judson V. Edwards, Abita Springs, LA (US); Nicolette T. Prevost, Prairieville, LA (US); Elena E. Graves, METAIRIE, LA (US); Joseph Dacorta, King George, VA (US); Dorne Yager, Mechanicsville, VA (US)

(21) Appl. No.: 17/713,803

(22) Filed: Apr. 5, 2022

Related U.S. Application Data
(60) Provisional application No. 63/171,171, filed on Apr. 6, 2021.

(51) Int. Cl. A61L 15/44; A61L 15/40; D06M 10/06; D06M 10/10; D06M 15/03; D06M 11/77; D06M 11/45

(52) U.S. Cl. CPC A61L 15/44; A61L 15/40; D06M 10/06; D06M 10/10; D06M 15/03; D06M 11/77; D06M 11/45; A61L 2400/04; A61L 2300/102

(57) ABSTRACT

The invention relates to fabric compositions with improved hemostatic properties comprising attached a zeolite, a zeolite/pectin complex, or a mixture thereof, and methods of preparing such fabric compositions.

### CROSS-REFERENCE TO RELATED APPLICATIONS

This application claims the benefit of U.S. Provisional Patent Application No. 63/171,171, filed Apr. 6, 2021, which is incorporated herein by reference in its entirety.

### FIELD OF THE INVENTION

The invention relates to fabric compositions comprising attached a zeolite, or a zeolite/pectin complex. The fabric compositions of the invention present with improved hemostatic properties.

### BACKGROUND OF THE INVENTION

The present invention was made as a result of activities undertaken with the scope of a research agreement. The parties to the research agreement were H&H Medical Corporation and United States Department of Agriculture, Agricultural Research Service.

Half of all deaths on the battlefield are caused by uncontrolled hemorrhage. In addition, high blood loss can lead to hypothermia, multiple organ failure, and infection. Thus, rapid hemostasis is essential for survival and recovery. The development of improved hemostatic agents for use in lethal extremity arterial hemorrhages has increased over recent years. The U.S. Army Institute for Surgical Research (USISR) and the Uniformed Services University of the Health Sciences has outlined ideal properties needed in a battlefield dressing. These include the following properties: (1) being able to rapidly stop large vessel arterial and venous bleeding two minutes after application when applied to an actively bleeding wound through a pool of blood; (2) no requirement for mixing or preapplication preparation; (3) simplicity of application by wounded victim, buddy, or medic; (4) light weight and durable; (5) long shelf life in extreme environments; (6) safe to use with no risk of injury to tissues or transmission of disease; and (7) inexpensive.

In addition, the design of a Prolonged Field Care (PFC) dressing has the following attributes: (1) affordable at a price point comparable to current cellulose wound packing material and compliant with the Berry Amendment and U.S. Trade statues, and suitable for individual carry; (2) no requirement for mixing or pre-application preparation; (3) acts as a barrier to microbial contamination and reduces bacterial colony formation; (4) can remain in place for 72-96 hours without tissue breakdown, reducing the need for frequent dressing changes; (5) conserves tissue viability by providing a moist environment, 6) prevents premature wound closure and formation of fistulae; (7) supports atraumatic removal by low adherence to tissue; and (8) reduced shedding of particular matter into wound bed, (9) five year shelf life in military relevant environment. Here the inventors put forward a dressing design that addresses hemorrhage control. The mechanisms of accelerated clotting of these dressings are commensurate with hemorrhage control efficacy and may halt blood flow within two minutes upon application. A recent review of prehospital hemorrhage control dressings has detailed the relative efficacy and safety properties of dressings. Disclosed here are detail and approach used for adhering zeolite which binds to greige cotton fiber in a unique way when formulated with pectin. The design of cotton fiber-adhered zeolite provides a route to non-egressing procoagulant.

Most of the current literature tends to characterize hemostatic textile-based materials as addressing hemorrhage control. Hemostatic material types may be viewed as accelerating surface hemostasis in the categories of untreated or treated textiles materials. Treated dressings are typically woven or nonwoven textile materials that have a hemostatic agent incorporated i.e. clay minerals, chitosan (used singularly as a fiber or coating), modified polysaccharides and fibrin sealant as the active clotting agent. Moreover, these types of dressings, which contain hemostasis-activating agents, have been classified as either factor concentrators, procoagulants, or mucoadhesives based on their mechanism of action to initiate and sustain blood coagulation. On the other hand, dressings which demonstrate hemostatic activity based on de novo design at the fiber level of textiles have been scarce, but there are some reports that are exemplary of this approach for single or multiple fiber blends (Fischer, T. H., et al., Journal of biomedical materials research. Part B, Applied biomaterials, 91(1): 381-389 (2009); Edwards, J. V., et al., J. Funct. Biomater., 5: 273-287 (2014); Edwards, J. V., and N. Prevost, Journal of Functional Biomaterials, 2(4): 391-413 (2011)).

Thus, fabric compositions with improved hemostatic properties, and highly effective, low-cost, and environmentally-friendly methods for preparing such fabric compositions are urgently needed.

Thus, fabric compositions with improved hemostatic properties, and new, highly effective, low-cost, and environmentally friendly methods for preparing such fabric compositions are urgently needed.

### SUMMARY OF THE INVENTION

Provided herein are fabric compositions comprising attached a zeolite, or a zeolite/pectin complex, and simple and low-cost approaches to preparing such fabric compositions.

In an embodiment, the invention relates to fabric compositions comprising attached a zeolite, a zeolite/pectin complex, or a mixture thereof.

In some embodiments of the invention, the fabric composition having attached a zeolite, zeolite/pectin complex, or a mixture thereof is a cloth, a woven fabric, a knitted fabric, a nonwoven fabric, or a final article. In some embodiments of the invention, the fabric composition having attached a zeolite, zeolite/pectin complex, or a mixture thereof is a single layered nonwoven fabric or a multilayered nonwoven fabric. In some embodiments of the invention, the fabric composition having attached a zeolite, zeolite/pectin complex, or a mixture thereof is a single layered fabric comprising about 5% by weight to about 95% by weight non-scoured, non-bleached greige cotton fibers; about 5% by weight to about 95% by weight bleached cotton fibers; about 5% by weight to about 60% by weight hydrophobic fibers; all percentages adding up to 100 wt %. In some embodiments of the invention, the fabric composition having attached a zeolite, zeolite/pectin complex, or a mixture thereof comprises about 60% by weight non-scoured, non-bleached greige cotton fibers, about 20% by weight bleached cotton fibers, and about 20% by weight hydrophobic fibers.

In some embodiments of the invention, the fabric composition having attached a zeolite, zeolite/pectin complex, or a mixture thereof is a multi-layered nonwoven fabric composition, comprising at least one inner layer containing about 50% by weight to about 95% by weight non-scoured, non-bleached greige cotton fibers and about 5% by weight to about 50% by weight hydrophobic fibers, all percentages adding up to 100 wt %, and at least one outer layer containing about 5% by weight to about 95% by weight non-scoured, non-bleached greige cotton fibers, about 5% by weight to about 95% by weight bleached cotton fibers, and about 5% by weight to about 60% by weight hydrophobic fibers, all percentages adding up to 100 wt %.

In an embodiment, the invention relates to an article of manufacture prepared with a fabric composition having attached a zeolite, zeolite/pectin complex, or a mixture thereof of the invention. In some embodiments of the invention the article of manufacture is a medical textile. In some embodiments of the invention the medical textile is a surgical arena fabric, a surgical personnel protective garment, a wound patient dressing, a non-wound patient dressing, a bandage, a gauze, a packing, or a cleaning material.

In an embodiment, the invention relates to a method for preparing a fabric composition comprising attached a zeolite, a zeolite/pectin complex, or a mixture thereof. The method comprises saturating a fabric composition with treatment solution; padding the saturated fabric composition; drying the padded fabric composition at a first temperature, and curing the dried fabric composition at a second, higher temperature. In some embodiments the method further comprises saturating the padded fabric composition with pectin, a zeolite, a pectin/zeolite complex. In some embodiments, the method comprises saturating the fabric composition with calcium chloride solution in step, followed by spraying pectin, a zeolite, a pectin/zeolite complex, or a mixture thereof on both sides of the saturated fabric composition, and drying the sprayed fabric composition.

### BRIEF DESCRIPTION OF THE DRAWINGS

FIG. 1A and FIG. 1B depict scanning electron microscopy (SEM) images of hydroentangled cotton fibers treated with zeolite/pectin. The image in FIG. 1A was taken at a lower magnification than the image in FIG. 1B. Size bars are present at the bottom of the images.

FIG. 2 depicts a graph of the clotting performance of formulations with zeolite and pectin. The results were obtained using a Lee White clotting assay. The Y Axis presents the mean of the time to clot formation in minutes. The X axis presents the samples, where: 6 is TACGauze treated with 1% PEC:NaY 1:10; 7 is TACGauze treated with NaY in H₂O 10 mg/mL; 11 is TACGauze treated with NaY in H₂O 100 mg/mL; 16 is TACGauze treated with 3% PEC:NaY 1:10; Control is TACGauze with no treatment; Procoagulant is Combat Gauze without additional treatment; Nothing: no fabric was added to the tube. Mean results of five separate runs are presented. ANOVA followed by Multiple Comparisons versus Control Group (Holm-Sidak method) revealed significant differences (p≤0.05) for all groups.

FIG. 3 depicts a schematic of a pad dry method of the invention for zeolite or pectin/zeolite complex application.

FIG. 4 depicts a pad-dry-spray method of the invention for zeolite or zeolite/pectin complex application.

### DETAILED DESCRIPTION

The present invention relates to fabric compositions with improved hemostatic properties, and simple and low-cost approaches to preparing such fabric compositions. The fabric compositions of the invention comprise attached zeolite, a zeolite/pectin complex, or a mixture thereof.

In an embodiment, the invention relates to finishing chemistries applied to cotton textiles to produce fabric compositions with improved hemostatic properties.

In an embodiment, the invention relates to fabric compositions comprising a zeolite, a zeolite/pectin complex, or a mixture thereof, where the zeolite and/or zeolite/pectin complex is attached to at least a portion of the fabric using traditional finishing chemistry.

In some embodiments of the invention, the fabric composition comprising a zeolite, or a zeolite/pectin complex attached to at least one portion of the fabric is a cloth, a woven fabric, a knitted fabric, a nonwoven fabric, or a final article.

In some embodiment, the invention relates to single layered fabric compositions comprising about 5% by weight to about 95% by weight non-scoured, non-bleached greige cotton fibers; about 5% by weight to about 95% by weight bleached cotton fibers; about 5% by weight to about 60% by weight hydrophobic fibers; all percentages adding up to 100 wt %; where the fabric composition comprises a zeolite, or a zeolite/pectin complex attached to at least one portion of the fabric. In some embodiments of the invention, the fabric composition comprising a zeolite, or a zeolite/pectin complex attached to at least one portion of the fabric comprises about 30% by weight to about 80% by weight non-scoured, non-bleached greige cotton fibers; about 20% by weight to about 70% by weight bleached cotton fibers; about 5% by weight to about 50% by weight hydrophobic fibers, all percentages adding up to 100 wt %. In some embodiments of the invention, the fabric composition comprising a zeolite, or a zeolite/pectin complex attached to at least one portion of the fabric comprises about 50% by weight to about 60% by weight non-scoured, non-bleached greige cotton fibers; about 20% by weight to about 30% by weight bleached cotton fibers; about 5% by weight to about 20% by weight hydrophobic fibers, all percentages adding up to 100 wt %. In some embodiments of the invention, the fabric composition comprising a zeolite, or a zeolite/pectin complex attached to at least one portion of the fabric comprises about 60% by weight non-scoured, non-bleached greige cotton fibers; about 20% by weight bleached cotton fibers; about 20% by weight hydrophobic fibers. In some embodiments of the invention, the fabric composition comprising a zeolite, or a zeolite/pectin complex attached to at least one portion of the fabric comprises about 85% by weight non-scoured, non-bleached greige cotton fibers, and about 15% by weight bleached cotton fibers.

In an embodiment, the invention relates to multi-layered fabric compositions containing at least one inner layer and at least one outer layer, and comprising a zeolite, a zeolite/pectin complex, or a mixture thereof attached to at least one portion of the fabric composition. In an embodiment of the invention, the multi-layered fabric composition comprising a zeolite, a zeolite/pectin complex, or a mixture thereof attached to at least one portion of the fabric composition contains (1) at least one inner layer containing (a) about 50% by weight to about 95% by weight (e.g., 50% to 95) non-scoured, non-bleached greige cotton fibers (preferably about 60% by weight to about 80% by weight (60-80); more preferably about 50% by weight to about 60% by weight (50-60)) and (b) about 5% by weight to about 50% by weight (e.g., 5% to 50%) hydrophobic fibers (preferably about 20% by weight to about 40% by weight (20-40), more preferably about 40% by weight to about 50% by weight (40-50)), all percentages adding up to 100 wt %, and (2) at least one outer layer containing (a) about 5% by weight to about 95% by weight (e.g., 5% to 95%) non-scoured, non-bleached greige cotton fibers (preferably about 30% by weight to about 80% by weight (30-80), more preferably about 50% by weight to about 60% by weight (50-60)), (b) about 5% by weight to about 95% by weight (e.g., 5% to 95%) bleached cotton fibers (preferably about 20% by weight to about 70% by weight (20-70), more preferably about 20% by weight to about 30% by weight (20-30)), and (c) about 5% by weight to about 60% by weight (e.g., 5% to 60%) hydrophobic fibers (e.g., polypropylene, nylon) (preferably about 5% by weight to about 50% by weight (5-50), more preferably about 5% by weight to about 20% by weight (5-20)); all percentages adding up to 100 wt %; wherein a zeolite, a zeolite/pectin complex, or a mixture thereof is attached to at least one portion of the fabric composition.

In an embodiment of the invention, at least one pectin/zeolite complex may be attached to at least one portion of the fabric composition by salts of earth metal ions, polycarboxylic acids, or acrylic acid. The zeolite may be any known natural or synthetic zeolite. Host guest molecules in the zeolites (ion-exchanged zeolites) include, for example, alkaline earth and transition metal ions, hydrogen, calcium, sodium, potassium, ammonia (ammonium ion), silver, titanium, zinc, copper, and iron (S. Chen et al., 2018, “Superior ion release properties and antibacterial efficacy of nanostructured zeolites ion-exchanged with zinc, copper, and iron,” RSC Adv. 8(66): 37949-37957).

The pectin may be any known pectin, such as a high methoxyl pectin, a low methoxyl pectin, an amidated pectin, or a mixture thereof.

A dilute solution of polyacrylic acid (or polyethylene glycol) may be employed to coat or embed the zeolite pectin complex at a facile interface between the fiber and point of application sufficient to promote contact of the zeolite and zeolite/pectin aggregates to the fabric while allowing exposure to the wound bed or trauma site. The added formulation ingredient may be applied in a spray or padding solution at an infinitesimal dilute level to afford activation of the blood coagulation pathway by the zeolite complex and platelets associated mechanism of blood clotting. Such an application may characterized as a nano-spray as well.

In some embodiments of the invention, the cellulosic portion of a fabric composition comprising a zeolite, a zeolite/pectin complex, or a mixture thereof attached to at least one portion of the fabric composition is from cotton, flax, hemp, jute, ramie, pineapple leaf, or abaca.

In an embodiment, the fabric composition comprising attached a zeolite, a zeolite/pectin complex, or a mixture thereof of the invention is a felted fabric, a woven fabric, a knitted fabric, a film-based composite, a nonwoven fabric, or a final article. Methods for preparing a fabric composition are known in the art.

A single layered nonwoven fabric composition may be prepared by any method known in the art. For example, needle punched webs of the different fiber blends may be prepared. Then the needle-punched webs of the different fiber blends may be uniformly hydroentangled using, for example, a Fleissner MiniJet system where the system is equipped with one low water pressure jet head that wets the incoming feed web material on its top face, while two high water pressure jet heads alternatively impact the wetted substrate on either face. For all the fabrics, the low water pressure head may be set to inject the water at about 30 bars, and the two high water pressure heads may be set at about 60 to about 100 bars (e.g., 60 to 100). A 23-mesh screen or lower may be employed to modulate the fabric fenestration. The fabric production speed may be about 5 m per minute. The resulting hydroentangled fabric is dried (e.g., using a meter-wide, gas-fired drum dryer) and may be wound onto a tube (e.g., cardboard) to form a compact fabric roll.

A significant amount of the cotton fiber cuticle and primary cell wall components are retained during hydroentanglement, but it is expected that increasing pressure removes more of the non-cellulosic fiber components. The non-cellulosic components can potentially detach or be removed from the fiber matrix due to the force of the water jets that creates an entangled fiber network and also exerts pressure, shear and friction on the outer cuticle layer of the fiber to an extent that this hydrophobic component (contains waxes) of the fiber begins to loosen or even detach from the secondary cell wall of the fiber. The inventors hypothesized that these cotton fiber components, which are partially retained from the hydroentanglement process, also play a role in the hemostatic activity of the fabric compositions (e.g., wound dressing material) since the hydrophobicity afforded by the waxes creates a negatively charged surface conducive to clotting acceleration.

As noted above, the fabric compositions of the invention may further contain zeolite and pectin. Pectin is utilized to adhere the zeolite to the fabric. The addition of zeolite and pectin to the fabric compositions may be achieved by any method known in the art, and one example is shown below.

In some embodiments of the invention there are multi-layered fabric compositions which contain two or three layers or more. In some embodiments of the invention the multilayered fabric composition comprises at least one nonwoven layer. The at least one nonwoven layer in the multi-layered fabric composition of the invention may be an inner layer or an outer layer. In some embodiments of the invention, a fabric composition contains at least one layer containing about 50% by weight non-scoured, non-bleached greige cotton fibers and about 50% by weight hydrophobic fibers, and at least one layer containing about 30% by weight non-scoured, non-bleached greige cotton fibers, about 50% by weight bleached cotton fibers, and about 20% by weight hydrophobic fibers. Methods of preparation of such multi-layers nonwoven fabric compositions are well known in the art.

The textiles or fabric compositions of the invention may be comprised of finishes that contain the hemostatic-active form of zeolite and an aluminosilicate hosting sodium, calcium, (and other earth metal ions of monovalent or divalent charge) hydrogen and ammonia to initiate accelerated formation of fibrin and clot formation upon contact with blood alone and when attached to the fabric. Zeolite is applied to the fabric as an emulsion at fabric:zeolite weight ratios of 1:1 to 1:40 suspensions and in combination with pectin (0.25% to 1% suspensions) and, calcium salts (from 1-5% solution). The application of the formulations is by pad dry cure or spay on delivery. The zeolite fabric compositions fare effective in promoting clot formation.

The fabric compositions comprising attached a zeolite, or a zeolite/pectin complex of the invention may be a yarn, a thread, a twine, a rope, a cloth, a woven fabric, a knitted fabric, a film-based composite, a nonwoven fabric, or a final article. In some embodiments of the invention, the fabric compositions comprising attached a zeolite, a zeolite/pectin complex, or a mixture thereof are a medical textile such as a surgical arena fabric, a surgical personnel protective garment, a wound or non-wound patient dressing, a bandage, a gauze, a packing, or a cleaning material.

A fabric composition of this invention may be a nonwoven fabric, which contains greige cotton along with other hydrophilic and hydrophobic fibers, the combination of which can produce rapid clotting as defined by both thromboelastography (TEG) and in vitro clotting experiments. When these fabric compositions are treated with a pectin/zeolite/calcium chloride formulation they produce a more rapid clotting response, sufficient to be considered a hemorrhage control dressing material.

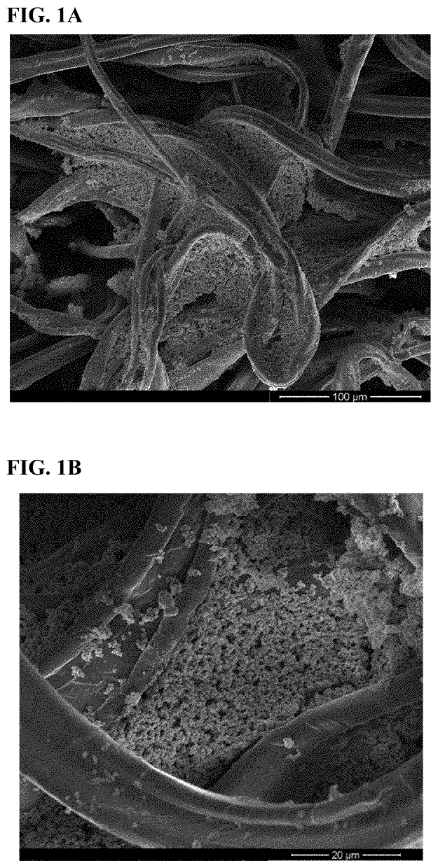

Greige cotton refers to unfinished cotton fibers that have not been scoured and bleached. The potential to use greige cotton in nonwoven absorbent products has received increased attention based on innovations in cotton cleaning and nonwovens processes that open and expose the hydrophilic cellulosic component of greige cotton fiber to water absorption. FIG. 1A and FIG. 1B portray micrographs of hydroentangled greige cotton fibers treated with pectin and zeolite. These figures show how the outer layers of the fibers are loosened or lifted from the fiber during the hydroentanglement process, resulting in a structure that enables embedded deposition of zeolite. Moreover, when pectin and an alkaline earth metal are applied with zeolite to greige cotton, pectin/zeolite complexes form that adhere between the cotton fibers. The alkaline earth metal may be beryllium, magnesium, calcium, strontium, barium, or radium.

Hydrophobic fibers include TRUECOTTON which is a non-scoured, non-bleached 100% natural greige cotton fiber which has been carefully mechanically cleaned to unprecedented levels. Since the cotton fiber has not been chemically altered, the natural waxes and oils remain on the fiber which allows for exceptional processing characteristics in any textile or nonwoven staple fiber manufacturing scheme. TRUECOTTON fiber is naturally hydrophobic, which sets it apart from any cotton fiber previously used for consumer goods. TRUECOTTON is 99.99% pure, meaning that 99.99% of foreign matter (e.g., cotton harvest contaminants in the form of cotton leaves, stems, and bracts; in other words, foreign matter includes anything in the way of trash that is carried over from the field to the ginning process) has been removed. The staple fiber length is about 19 to about 30 mm, hydrophobicity reflected in the water contact angle which is 140.9°+5.3, and has a denier (micronaire) of about 3.5 to about 5.5 (e.g., 3.5 to 5.5; preferably about 4.0 to about 5.5 (e.g., 4.0 to 5.5)). Other hydrophobic fibers similar to TRUECOTTON may be used.

Other components (e.g., other hydrophilic or hydrophobic components) known in the art may be added to the fabric compositions of the invention provided they do not substantially interfere with the intended activity and efficacy of the fabric compositions; whether or not a compound interferes with activity and/or efficacy can be determined, for example, by the procedures utilized below. Hydrophilic fibers include, for example, bleached and scoured cotton, polyurethane, rayon, spandex, polyacrylate, flax, hemp, ramie, bamboo, alginate, chitosan, hyaluronan, regenerated cellulose, N-acetylglucosamine, and carboxymethylcellulose. Hydrophobic fibers include, for example, polyolefin, polyester, polyacrylate, wool, glass filament, collagen, polypropylene, and nylon.

The terms “fabric” and “textile are used interchangeably herein, and refer to a cloth, a woven fabric, a knitted fabric, a nonwoven fabric, or an article of manufacture. The article of manufacture may be a medical textile such as a surgical arena fabric, a surgical personnel protective garment, a wound dressing, a non-wound dressing, a bandage, a gauze, a packing, a cleaning material, or a face mask.

The terms “wound dressing”, “wound plaster”, “wound bandage” or “wound covering” are used interchangeably herein, and describe dressings for topical application onto external wounds, in order to prevent penetration of foreign bodies into the wound and to absorb blood and wound secretions. Wound dressings are not limited to a particular size or shape. A wound dressing may be a single layer fabric composition, or may be a multi-layered fabric composition. For example, a wound dressing may be in the form of a trilayer fabric composition, comprising two outer layers and an inner layer. A multilayer wound dressing fabric composition has been described herein as comprising first, second and third layers, although it may comprise further layers, such as fourth, fifth, sixth, seventh, eighth, ninth, tenth layers, or more. The further layers may comprise any of the features referred to herein in relation to the inner and outer layers. This also applies to fabric compositions in general.

Hemostatic Formularies and Their Activity: Formulations are categorized by the function they impart to the dressing, —hemostatic control only, and both antimicrobial and hemostatic control. Similar reagents are used though their purpose may differ in certain formulations.

Pectin in all formulations was employed to promote adherence of created microparticles or added zeolites to the cotton fiber surface. Calcium chloride and sodium carbonate were used to create calcium carbonate microparticles for hemorrhagic control. Calcium chloride was also used as a source of calcium ions to induce clotting. In formulations with only calcium salt and zeolite, calcium cation can exchange with the sodium cations as counter cations to the aluminosilicate cage framework of the faujasite Y zeolite.

Tables 2 to 5 summarize the thromboelastography(TEG) results of the Y zeolite formulations with both sodium (NaY) and ammonium (NH₄Y), as counter cations, applied in various formulations to TACGauze with pad-dry-cure application method. Consistently, zeolite alone or zeolite with added calcium adsorbed on fabric performed similar to the procoagulant with a time to start clot formation (R) at about 4.2-5.6 and the speed of clot formation (K) at less than 2.4 minutes, similar to procoagulants and commensurate with hemorrhage control. Pectin at 0.25-0.5% with zeolite alone or in combination with calcium also performed favorably with 4.8-5.5 reaction times. In Table 5 it can be seen that the application of ammonium Y zeolite with pectin formularies gave comparable procoagulant results. The role of adhering zeolite to the cotton fibers is portrayed in FIG. 1A and FIG. 1B which show the Scanning electron microscopy (SEM) images showing the morphological changes of the modified cotton fibers. As shown in FIG. 1A and FIG. 1B, the formulations with pectin and zeolite resulted in aggregates of zeolite that form pectin-zeolite complexes deposited as ordered inter-fibrillar sheets. Zeolite aggregates are composed of submicron to micron sized particles attached to the cotton fiber by way of pectin in the cotton primary cell. Pectin is characterized as forming the classic ‘egg-box’ structure promoting adhesion of zeolite. Thus, observed are the ordered aggregates of submicron particles (zeolite/pectin) observed between the fibers of the fabric.

Table 6 summarizes the thromboelastography (TEG) results of cotton fabrics treated with the second application method, pad-spray-dry, using the same formulary ingredients. It performed similarly to the pad-dry method. Notably an improved fabric hand was imparted. As seen in Table 6, two different cotton fabrics, TACGauze, cotton/polypropylene blend, and NW85, a cotton nonwoven fabric 85:15 greige cotton:bleached cotton, performed slightly better with the one step pad-dry method. The NW85 fabric formularies gave a decreased R value of approximately 1 minute. Similarly, the K value (time to clot formation) was decreased with formularies of higher add-on.

Hemostatic Antimicrobial Activity: Table 6 and Table 7 summarize TEG clotting results of the ascorbic acid crosslinked fabrics in combination with one and ten percent zeolite, and BIOgauze formulated with sodium zeolite and pectin, which demonstrated favorable clotting commensurate with hemorrhage control activity as shown in Table 7.

Table 6 summarizes some of the TEG clotting results of the ascorbic acid-crosslinked fabrics in combination with one and ten percent zeolite. However, this approach appears not to favor improved clotting profiles. On the other hand, BIOgauze formulated with sodium zeolite and pectin demonstrated favorable clotting commensurate with hemorrhage control activity as shown in Table 7.

As shown in Table 8 the combination of sodium zeolite with pectin is somewhat comparable to employing alginate. When sodium carbonate and calcium chloride were employed in the formulation with pectin the time to clot formation was generally within the range expected for a procoagulant but time to fibrin formation was somewhat slower. Table 9 shows that the use of calcium oxide did not improve on this trend. The use of spray applications to TACgauze showed comparable clotting times commensurate with procoagulant hemorrhage control.

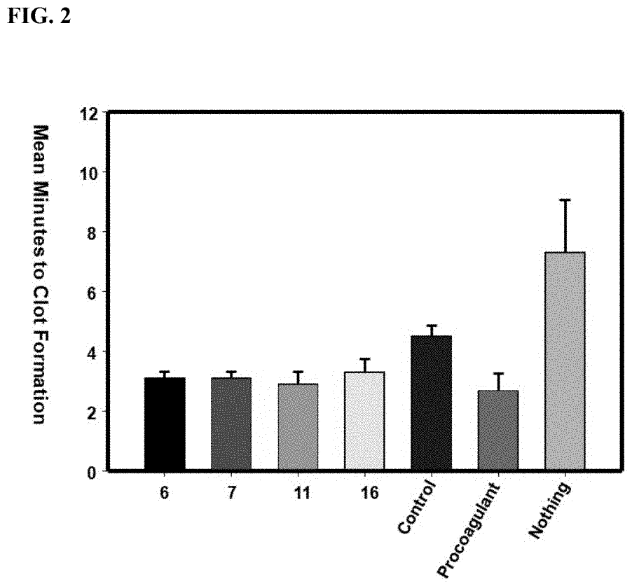

FIG. 2 demonstrates that clotting performance of formulations with zeolite and pectin perform similar to a hemorrhage control procoagulant when tested in a Lee White clotting assay where platelets are understood to be freshly obtained porcine blood.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

“Optional” or “optionally” means that the subsequently described event or circumstance may or may not occur, and that the description includes instances in which said event or circumstance occurs and instances where it does not. For example, the phrase “optionally comprising an antimicrobial agent” means that the fabric composition of the invention may or may not contain an antimicrobial agent and that this description includes fabric compositions that contain and do not contain an antimicrobial agent. Also, by example, the phrase “optionally adding an antimicrobial agent” means that the method may or may not involve adding an antimicrobial agent and that this description includes methods that involve and do not involve adding an antimicrobial agent.

Other compounds (e.g., antimicrobial agent) may be added to the fabric compositions of the invention provided they do not substantially interfere with the intended activity and efficacy of the fabric compositions; whether or not a compound interferes with activity and/or efficacy can be determined, for example, by the procedures utilized below.

By the term “effective amount” of a compound or property as provided herein is meant such amount is capable of performing the function of the compound or property for which an effective amount is expressed. As will be pointed out below, the exact amount required will vary from process to process, depending on recognized variables such as the compounds employed, and the processing conditions observed. Thus, it is not possible to specify an exact “effective amount.” However, an appropriate effective amount may be determined by one of ordinary skill in the art using only routine experimentation.

The amounts, percentages, and ranges disclosed herein are not meant to be limiting, and increments between the recited amounts, percentages, and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount.

As used herein, the term “about” is defined as plus or minus ten percent of a recited value. For example, about 1.0 g means 0.9 g to 1.1 g.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

Regarding double entries in the tables, there is either some slight variation in the formulation or the add-ons vary. Thus, duplicate entries in terms of samples tested.

All of the references cited herein, including Patents and Patent Application Publications, are incorporated by reference in their entirety. Also incorporated by reference in their entirety are the following references: Wagner, W., et al., J. Surgical Res., 66: 100-108 (1996); U.S. Pat. Nos. 6,809,231; 9,474,827; 9,463,119; U.S. Patent Application Publication Number 20170128270; U.S. Patent Application Publication Number 20190380878; U.S. patent application Ser. No. 16/110,169.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein). The invention illustratively disclosed herein suitably may be practiced in the absence of any element (e.g., method (or process) steps or composition components) which is not specifically disclosed herein. Thus, the specification includes disclosure by silence (“Negative Limitations In Patent Claims,” AIPLA Quarterly Journal, Tom Brody, 41(1): 46-47 (2013): “ . . . Written support for a negative limitation may also be argued through the absence of the excluded element in the specification, known as disclosure by silence . . . Silence in the specification may be used to establish written description support for a negative limitation. As an example, in Ex parte Lin [No. 2009-0486, at 2, 6 (B.P.A.I. May 7, 2009)] the negative limitation was added by amendment . . . In other words, the inventor argued an example that passively complied with the requirements of the negative limitation . . . was sufficient to provide support . . . This case shows that written description support for a negative limitation can be found by one or more disclosures of an embodiment that obeys what is required by the negative limitation . . . .”

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

The following examples are intended only to further illustrate the invention and are not intended to limit the scope of the invention as defined by the claims.

### EXAMPLES

Having now generally described this invention, the same will be better understood by reference to certain specific examples, which are included herein only to further illustrate the invention and are not intended to limit the scope of the invention as defined by the claims.

### Example 1

### Materials and Methods

Disclosed in this example are the materials and methods used herein to develop fabric compositions containing attached a zeolite, and/or a zeolite/pectin complex.

Thromboelastography: Citrated bovine blood was used for TEG analysis. Blood analysis: 340 μl bovine blood and 20 μl CaCl₂) were added to the sample cup and the run was begun. Alternatively, with fabric samples: 1 mg fabric was added to the sample cup in each channel with 20 μl citrated saline to wet the fabric. Then to each cup, 30 μL of 0.2 M CaCl₂) were added and followed by 310 μL of citrated bovine blood. Both channel runs were started immediately.

Preparation of Single Layered Nonwoven Material. Hydroentanglement of fibrous webs into nonwoven fabric structures: A commercially available bale of pre-cleaned greige cotton was acquired from T. J. Beall, LLC (Greenwood, Miss., USA). Polypropylene fibers were acquired commercially. A bleached version of TRUE COTTON raw cotton was also acquired from T. J. Beall. The needle punched webs of the different fiber blends were uniformly hydroentangled using a Fleissner MiniJet system. The system was equipped with one low water pressure jet head that wets the incoming feed web material on its top face while two high water pressure jet heads alternatively impact the wetted substrate on either face. For all the fabrics, the low water pressure head was set to wet the fabric at 30 bars of water pressure and the two high water pressure heads were set at either 60, 80, or 100 bars. The fabric production speed was 5 meters per minute. The resulting hydro-entangled fabric was dried using a meter-wide, gas-fired, through-Drum Dryer and wound onto a cardboard tube to form a compact fabric roll. The hydroentangling line utilizes municipal water that is passed through a reverse osmosis filter that is set to give a water hardness of 70 to 110 PPM.

Fabric Treatment. Materials and Reagents: CBV100 and CBV300 zeolites were purchased from Zeolyst International (Conshohoken, Pa., USA). They are the Synthetic faujasite Y zeolite with a cation, CBV100 has sodium cation (NaY), and CBV300 has ammonium cation (NH₄Y). The SiO₂/Al₂O₃ molar ratio for NaY is 4.9-5.4 and for NH₄Y is 5.1. All other chemicals and fabrics were from existing supply/inventory. The pectin (PEC) from citrus peel (>74% galacturonic acid) and calcium chloride (CaCl₂)) were purchased from Sigma Aldrich (now Millipore Sigma; Burlington, Mass., USA). Ultrapure water (18Ω), water was obtained by using a Milli-Q water purification system (Millipore-Sigma), and was used as solvent. The fabrics used were as follows: TACGauze (TGz) from H&H Medical Corporation (Williamsburg, Va., USA), a blend of 50% greige cotton/30% bleached cotton/20% polypropylene; Fine Mesh Gauze (FMGz), 100% bleached cotton (#4-2915 inside roll 36″ 50-yard roll from DeRoyal Industries (Powell, Tenn., USA); Hydroentangled nonwoven fabric (NW85), 85% true cotton (greige cotton) and 15% bleached cotton (true cotton) produced at Southern Regional Research center (SRRC) (New Orleans, La., USA). For spray application of formulation, an Aldrich-flask type thin-layer chromatography (TLC) sprayer was used.

Treatments done for Hemostatic Control only were: (1) Y Zeolite alone in water; (2) varying weight percent of pectin and powdered Y zeolite; (3) 1% CaCl₂) and varying percent powdered Y zeolite; (4) varying weight percent of pectin, CaCl₂) and powdered Y zeolite; (5) 0.5% Pectin, 0.5M sodium carbonate, 1% CaCl₂); and (6) 0.5% Pectin, 0.5M sodium carbonate, 1% CaCl₂) and varying percent of powdered NaY zeolite. The formulations were made with NaY and NH₄Y as the zeolite. Varying weight percent citric acid, sodium hypophosphite, pectin, CaCl₂), pharmaceutical grade acrylic acid, and powdered Y zeolite were used.

Treatments done for Hemostatic Control and Antimicrobial Activity were: (1) Varying weight percent citric acid, ascorbic acid, sodium hypophosphite and powdered Y zeolite; (2) Varying weight percent citric acid, ascorbic acid, sodium hypophosphite, pectin and powdered Y zeolite; (3) Varying weight percent citric acid, ascorbic acid, sodium hypophosphite, pectin, CaCl₂) and powdered Y zeolite. BIOGauze (or pretreatment of greige cotton/cotton blend with 0.95% (w/w) ascorbic acid & 0.6% (w/w) hexanol) then treated with either: (1) Y Zeolite alone in water; (2) varying weight percent of pectin and powdered Y zeolite; (3) 1% CaCl₂) and varying percent powdered Y zeolite; (4) varying weight percent of pectin, CaCl₂) and powdered Y zeolite; (5) 0.5% Pectin, 0.5M sodium carbonate, 1% CaCl₂); and (6) 0.5% Pectin, 0.5M sodium carbonate, 1% CaCl₂) and varying percent of powdered NaY zeolite. Formulations were made with NaY or NH4 Y as the zeolite.

The Y zeolite powder acts as a procoagulant and is used to impart accelerated hemostasis. Zeolite was used without any activation (removal of bound water in its cavity). Zeolite powder was added to solution after other reagents were dissolved or mixed. After addition, each solution was vortexed and then stirred until use to suspend the particles.

### Example 2

### Application of Zeolites

Two methods were originally used in the application of zeolite to the fabrics. Each method could have a variation of either one or two steps.

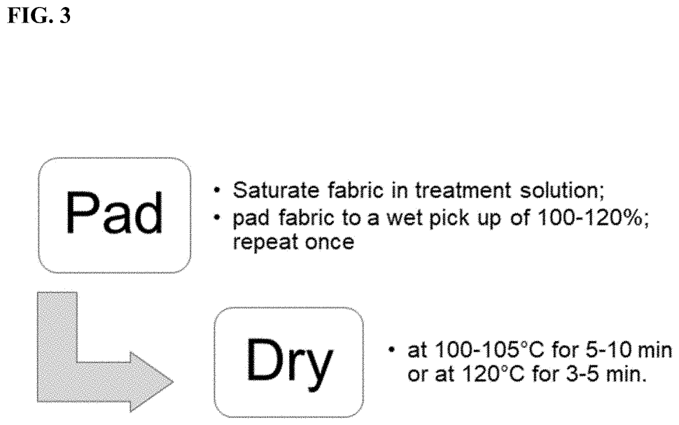

Application Method 1: Pad-Dry, a schematic diagram of which is shown in FIG. 3. To treat the fabrics, fabric swatches were submersed and saturated in a solution volume of 20 times the weight of fabric. The saturated swatches were padded with a CALLINGER hand-cranked wringer. Padding was repeated and the wet padded weight of the swatch was recorded. The swatches were dried on a screen or metal frame in a force draft oven of either 100° C. to 105° C. for 5 to 10 minutes or 120° C. for 3 to 5 minutes without tension. Swatches were not rinsed. The formulation swatches equilibrated overnight before weight measurement. In a two-step pad-dry method, the fabric swatches were padded with a weight percent solution of CaCl₂) and dried for 3 minutes at 100° C. and then padded with weight percent pectin and zeolite solution and dried at 105° C. or 120° C. for 5 minutes. Swatches were equilibrated overnight at ambient conditions and weighed.

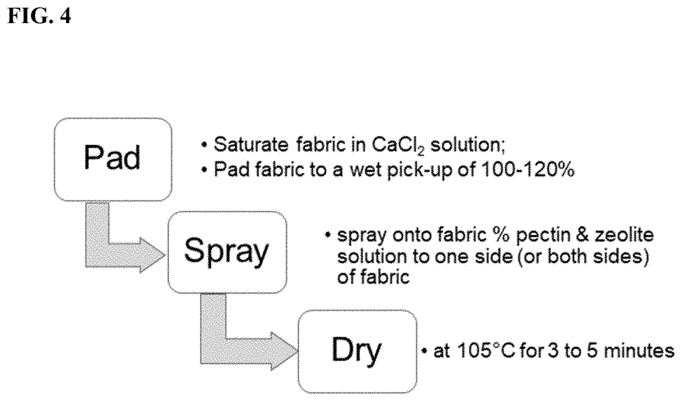

Application Method 2: Pad-Spray, a schematic diagram of which is shown in FIG. 4. Fabric swatches were saturated with a weight percent CaCl₂) solution volume 20 times its weight, and were padded to remove excess solution. Then, swatches were clipped to a frame and sprayed with a percent solution of pectin and zeolite previously vortexed using an aspirator (glass nozzle and flask attached to house air). Acrylic acid at low aqueous concentrations was also used as an adherent. Spraying application was in a sweeping motion and applied to single-side of two-sided fabric surface. The fabric formulation was dried for 3 minutes at 105° C. Without rinsing, fabrics were equilibrated overnight to room atmosphere and humidity and then weighed.

### Example 3

### Hemostatic Control and Antimicrobial Activities

The hemostatic control abilities and antimicrobial activities of greige cotton-containing-materials treated with ascorbic acid were tested.

Tables 1 to 4 summarize the thromboelastography (TEG) results of the Y zeolite formulations with both sodium (NaY) and ammonium (NH4Y), as counter cations, applied in various formulations to TACGauze with pad-dry-cure application method. Consistently, zeolite alone or zeolite with added calcium adsorbed on fabric performed similar to the procoagulant with a time to start clot formation (R) at about 4.2-5.6 and the speed of clot formation (K) at less than 2.4 minutes, and commensurate with hemorrhage control. Pectin at 0.25% to 0.5% with zeolite alone or in combination with calcium also performed favorably with 4.8 to 5.5 reaction times. Table 1 below presents values measured on two different dates/runs. Abbreviations: PEC=pectin, NaY=sodium Y zeolite, CaCl₂=calcium chloride, TACGz=TACGauze.

The results in this Example show that zeolite alone or zeolite with added calcium adsorbed on fabric consistently performed similar to the procoagulant commensurate with hemorrhage control. Pectin at 0.25% to 0.5% with zeolite alone or in combination with calcium also performed favorably with 4.8 to 5.5 reaction times.

TABLE 1
Percent add-on and TEG data of pad-dry applications on TAC Gauze

| | | % | R | | K | |
|---|---|---|---|---|---|---|
| Sample ID | Sample Description* | Add-on | (min) | σ | (min) | σ |
| 021919-1 | 1% Pectin only | 5.4 | 9.8 | | 6.8 | 9.8 |
| 021919-2 | 1% Pectin + 1% CaCl₂ | 12.0 | 10.7 | | 7.2 | 10.7 |
| 021919-3 | 1% PEC + 10% NaY | 43.6 | 6.6 | | 2.6 | 6.6 |
| 021919-4 | 1% PEC + 20% NaY | 81.1 | 5.1 | 0.2 | 2.3 | 0.1 |
| | | | 5.2 | | | |
| 021919-5 | 10% NaY only | 45.6 | 4.5 | 0.6 | 2.6 | 0.3 |
| | | | 5.3 | 0.1 | 3.4 | 0.6 |
| 021919-6 | 20% NaY only | 82.2 | 4.2 | 0.4 | 3.2 | 0.5 |
| | | | 7.2 | | 11.0 | |
| 021919-7 | 1% CaCl₂ only | 11.9 | 6.8 | | 4.2 | |
| 021919-8 | 1% PEC + 10% NaY + 1% CaCl₂ | 42.2 | 5.9 | | 4.1 | |
| 021919-9 | 1% PEC + 20% NaY + 1% CaCl₂ | 85.3 | 5.8 | | 4.4 | |
| 021919-10 | TACGz (untreated) | | 11.9 | | 7.3 | |
| 022819-5 | 0.5% PEC + 10% NaY | 45.7 | 4.8 | 0.4 | 2.6 | 0.0 |
| 022819-7 | 0.25% PEC + 0.25% NaY | 2.4 | 7.0 | 0.6 | 3.2 | 0.1 |
| 022819-8 | 0.25% PEC + 1% NaY | 5.5 | 6.2 | 0.3 | 3.4 | 0.2 |
| 022819-9 | 0.25% PEC + 2.5% NaY | 12.3 | 7.5 | 0.9 | 3.2 | 0.4 |
| 022819-10 | 0.25% PEC + 10% NaY | 43.4 | 4.8 | 0.0 | 3.0 | 0.8 |
| | Blood | | 12.5 | 1.1 | 5.3 | 0.5 |
| | | | 15.2 | 0.2 | 6.8 | 0.9 |
| | Procoagulant | | 3.8 | 0.0 | 2.1 | 0.4 |
| | | | 3.9 | | 4.0 | |

TABLE 2
Percent add-on and TEG data of pad-dry* application on TAC Gauze

| | | % | R | | K | |
|---|---|---|---|---|---|---|
| Sample ID | Sample Description* | Add-On | (min) | σ | (min) | σ |
| 031319-1 | 0.5% Na Alginate, 0.5M Na₂CO₃ + 1% CaCl₂ + | 13.0 | | | | |
| | 1% NaY | | | | | |
| 031319-2 | 0.5% Na Alginate, 0.5M Na₂CO₃ + 1% CaCl₂ + | 19.9 | | | | |
| | 10% NaY | | | | | |
| 031319-3 | 0.5% Pectin, 0.5M Na₂CO₃ + 1% CaCl₂ + 1% NaY | 12.7 | | | | |
| 031319-4 | 0.5% Pectin, 0.5M Na₂CO₃ + 1% CaCl₂ + | 18.4 | 6.7 | 0.6 | 2.6 | 0.5 |
| | 10% NaY | | | | | |
| 031319-5 | 0.5% Na Alginate, 0.5M Na₂CO₃ + 1% CaCl₂ only | 16.2 | | | | |
| 031319-6 | 0.5% Pectin, 0.5M Na₂CO₃ + 1% CaCl₂ only | 12.7 | | | | |
| 031319-7 | 1% CaCl₂ + 1% NaY only | 10.6 | | | | |
| 031319-8 | 1% CaCl₂ + 10% NaY only | 28.8 | 4.5 | 0.1 | 1.7 | 0.1 |
| 031519-1 | 0.5M Na₂CO₃, 1% CaCl₂ only | 11.4 | | | | |
| 031519-2 | 0.5M Na₂CO₃, 1% CaCl₂, 1% NaY | 9.9 | | | | |
| 031519-3 | 0.5M Na₂CO₃, 1% CaCl₂, 10% NaY | 31.9 | 5.4 | 0.4 | 2.0 | 0.3 |
| 031519-4 | 0.5M NaOH + 1% CaCl₂ only | 17.2 | | | | |
| 031519-5 | 0.5M NaOH, 1% CaCl₂, 1% NaY | 17.6 | | | | |
| 031519-6 | 0.5M NaOH, 1% CaCl₂, 10% NaY | 30.9 | 4.7 | 0.6 | 1.6 | 0.4 |
| | Blood | — | 12.7 | 2.2 | 4.9 | 0.4 |
| | Procoagulant | — | 3.3 | 0.0 | 1.4 | 0.1 |

*Stepwise reagent addition to pad bath. Soak, pad, then dry.
Abbreviations: Na₂CO₃ = sodium carbonate, NaOH = sodium hydroxide; CaCl₂ = calcium chloride; Na Alginate = alginic acid, sodium salt, NaY = sodium Y zeolite.

TABLE 3
Percent add-on and TEG data of pad-dry application of pectin, calcium
chloride (CaCl₂) and zeolite on TACGauze & fine mesh gauze

| | | % | R | | K | |
|---|---|---|---|---|---|---|
| Sample ID | Sample Description | Add-On | (min) | σ | (min) | σ |
| 082019-1 | TACGz 0.5% PEC + 2% CaCl₂ + 5% NaY (1) | 40.4 | 5.2 | 0.4 | 1.8 | 0.2 |
| 082019-2 | TACGz 2% CaCl₂, 2% PEC + 10% NaY (2) | 48.7 | 6.4 | 1.0 | 2.7 | 1.1 |
| 082019-3 | TACGz 5% CaCl₂, 2% PEC + 10% NaY (2) | 85.9 | 5.6 | 0.3 | 2.2 | 0.6 |
| 082019-4 | TACGz 5% CaCl₂, 1% PEC + 5% NaY (2) | 52.2 | 5.8 | 0.1 | 2.0 | 0.3 |
| 082119-1 | FMGz 0.5% PEC + 2% CaCl₂ + 5% NaY (1) | 22.4 | 6.8 | 0.5 | 3.9 | 0.3 |
| 082119-2 | FMGz 2% CaCl₂, 2% PEC + 10% NaY (2) | 29.4 | 6.8 | 0.1 | 4.6 | 1.1 |
| 082119-3 | FMGz 5% CaCl₂, 2% PEC + 10% NaY (2) | 37.2 | 7.2 | 0.0 | 3.0 | 0.4 |
| 082119-4 | FMGz 5% CaCl₂, 1% PEC + 5% NaY(2) | 21.2 | 7.4 | 0.6 | 4.6 | 1.1 |
| | TAC Gauze (untreated) | n/a | 8.1 | 0.6 | 3.8 | 0.1 |
| | Blood(bovine) | n/a | 14.9 | 1.4 | 6.2 | 1.3 |
| | Procoagulant | n/a | 3.4 | 0.2 | 1.1 | 0.1 |

(1) = one-step pad-dry.
(2) = two-steps: CaCl₂ padded and dried then padded with pectin + zeolite and dried; (S) = sprayed application of zeolite; TACGz = TACGauze, FMGz = fine mesh gauze, bleached 100% cotton, PEC = pectin, NaY = sodium Y zeolite

In Table 4, it can be seen that the application of ammonium Y zeolite with pectin formularies gave comparable procoagulant results. The role of adhering zeolite to the cotton fibers is portrayed in FIG. 1A and FIG. 1B which show the Scanning electron microscopy (SEM) images showing the morphological changes of the modified cotton fibers. As shown in these figures, the formulations with pectin and zeolite resulted in aggregates of zeolite that form pectin-zeolite complexes deposited as ordered inter-fibrillar sheets. Zeolite aggregates are composed of submicron to micron sized particles attached to the cotton fiber by way of pectin in the cotton primary cell. Pectin is characterized as forming the classic ‘egg-box’ structure promoting adhesion of zeolite. Thus, observed are the ordered aggregates of submicron particles (zeolite/pectin) observed between the fibers of the fabric.

TABLE 4
Percent add-on and TEG data of pad-dry application
of pectin, CaCl₂ and NH₄Y zeolite on TAC Gauze

| | | % | R | | K | |
|---|---|---|---|---|---|---|
| Sample ID | Description | Add-On | (min) | σ | (min) | σ |
| 071619-1 | 10%(w/v) NH₄Y only | 33.8 | 5.1 | 0.8 | 2.2 | 1.0 |
| 071619-2 | 0.1M CaCl₂ + 10% NH₄Y | 35.3 | 5.7 | 0.3 | 3.2 | 1.5 |
| 071619-3 | 0.25% PECTIN + 10% NH₄Y | 36.2 | 5.4 | 0.3 | 2.8 | 0.8 |
| 071619-4 | 0.5% PECTIN + 10% NH₄Y | 37.5 | 5.2 | 0.4 | 2.4 | 0.5 |
| 071619-5 | 1.0% PECTIN + 10% NH₄Y | 41.6 | 6.5 | 0.4 | 2.6 | 1.0 |
| 071619-6 | 0.5% PEC + 0.1M CaCl₂ + 10% NH₄Y | 46.7 | 5.9 | 0.5 | 2.0 | 0.3 |
| | TACGauze (untreated) | | 14.4 | | 6.5 | |
| | Blood(bovine) | | 19.1 | 1.8 | 10.7 | 2.3 |
| | Procoagulant | | 5 | 0.3 | 2.4 | 0.7 |

PEC = pectin, NH₄Y = Ammonium Y zeolite; CaCl₂ = calcium chloride

Table 5 summarizes the thromboelastography (TEG) results of cotton fabrics treated with the second application method, pad-spray-dry, using the same formulary ingredients. It performed similarly to the pad-dry method. Notably an improved fabric hand was imparted. As seen in table 6 (5), two different cotton fabrics, TACGauze, cotton/polypropylene blend, and NW85, a cotton nonwoven fabric 85:15 greige cotton:bleached cotton, performed slightly better with the one step pad-dry method. The NW 85 fabric formularies gave a decreased R value of approximately 1 minute. Similarly, the K value (time to clot formation) was decreased with formularies of higher add-on.

TABLE 5
Percent add-on and TEG data of pad-spray application of
CaCl₂, pectin, and zeolite on TAC Gauze & Nonwoven cotton fabric

| | | % | R | | K | |
|---|---|---|---|---|---|---|
| Sample ID | Description | Add-On | (min) | σ | (min) | σ |
| 103019-1 | TACGz 0.5% PEC + 2% CaCl₂ + | 35.4 | 5.7 | | 2.1 | |
| | 5% NaY^{≠} | | 6.6 | 0.2 | 1.5 | 0.1 |
| 103019-2 | TACGz 1% CaCl₂; 0.5% PEC + 5% NaY (S) | 34.3 | 6.5 | 1.0 | 2.2 | 0.5 |
| | | | 6.4 | 0.5 | 1.6 | 0.3 |
| 103019-3 | TACGz 2% CaCl₂; 0.5% PEC + 5% NaY (S) | 34.9 | 6.4 | 0.8 | 2.3 | 1.3 |
| | | | 6.5 | 0.4 | 2.1 | 0.6 |
| 103019-4 | TACGz 5% CaCl₂; 0.5% PEC + 5% NaY (S) | 50.3 | 6.8 | | 2.3 | |
| | | | | 0.7 | 1.4 | 0.5 |
| 110419-7 | TACGz 2% CaCl₂; 1% PEC + 5% NaY(S) | 36.3 | 6.2 | 0.5 | 2.3 | 0.6 |
| 110419-8 | TACGz 5% CaCl₂; 1% PEC + 5% NaY(S) | 96.5 | 6.1 | 0.3 | 2.2 | 0.0 |
| | TACGauze (untreated) | n/a | 9.2 | | 3.4 | |
| | | | 15.4 | 0.1 | 6.0 | 0.6 |
| | NW85 (untreated) | | 9.2 | | 7.6 | |
| 110419-9 | NW85 2% CaCl₂; 1% PEC + 5% NaY(S) | 37.7 | 7.2 | 1.4 | 3.7 | 0.6 |
| 110419-10 | NW85 5% CaCl₂; 1% PEC + 5% NaY(S) | 104.8 | 5.0 | | 1.3 | |
| | Blood(bovine) | n/a | 12.8 | 0.4 | 4.0 | 0.2 |
| | | | 17.3 | 1.2 | 6.5 | 1.9 |
| | Procoagulant | n/a | 4.2 | | 2.3 | |
| | | | 4.0 | 0.2 | 0.8 | 0.0 |
| 101819-1 | NW85 1% CaCl₂; 0.5% PEC + 5% NaY(S) | 25.3 | 6.8 | 0.2 | 2.2 | 0.1 |
| 101819-2 | NW85 2% CaCl₂; 0.5% PEC + 5% NaY(S) | 33.6 | 7.2 | 0.1 | 3.6 | 1.9 |
| 101819-3 | NW85 5% CaCl₂; 0.5% PEC + 5% NaY(S) | 54.1 | 5.1 | 0.2 | 1.7 | 0.4 |
| 101819-4^{≠} | NW85 0.5% PEC + 2% CaCl₂ + | 42.8 | 5.6 | 0.2 | 2.2 | 0.0 |
| | 5% NaY^{≠} | | | | | |
| | NW85(untreated) | n/a | 11.3 | 0.4 | 6.0 | 0.6 |
| | Blood(bovine) | n/a | 13.8 | 0.5 | 3.5 | 0.5 |
| | Procoagulant | n/a | 3.6 | 0.1 | 1.4 | 0.1 |

*In instances where the measurements were done during a different date/run, numbers for each run are on the same line.
Abbreviations: PEC = pectin, NaY = sodium Y zeolite, CaCl₂ = calcium chloride, NW85 = Hydroentangled nonwoven 85% greige cotton and 15% bleached cotton (HE0224R5); TACGz = TACGauze (S) = sprayed application of zeolite in pectin solution;
^{≠}padded one step application of formulation.

### Example 4

### Antiviral Properties of Formulations with Zeolite

Hemostatic and Antimicrobial. Crosslinking: ascorbic acid and zeolite formulations: Four 40 mL solutions were made to treat the swatches. They were as follows: (1) 7% (w/v) citric acid (CA) and 4.8% (w/v) sodium hypophosphite monohydrate (SHP) (NaH₂PO₂.H₂O); (2) 7% (w/v) CA and 4.8% (w/v) NaH₂PO₂.H₂O, and 1% (w/v) ascorbic acid (Asc.A.), ˜54 mM; (3) 7% (w/v) CA and 4.8% (w/v) NaH₂PO₂.H₂O, 1% (w/v) Asc. A. and 1% (w/v) Sodium Y zeolite (NaY); (4) 7% (w/v) CA and 4.8% (w/v) NaH₂PO₂.H₂O, 1% (w/v) Asc. A. and 10% (w/v) NaY. Swatches were saturated, padded and dried for 3 minutes at 95° C.; then, they were cured for 2 minutes at 160° C. All swatches were then rinsed with deionized water. They were padded to remove excess water and dried in oven at 100° C. for 3 minutes. They were weighed after equilibrating overnight.

Antimicrobial Formulary Hemostatic Activity: Table 6 and Table 7 summarize TEG clotting results of the ascorbic acid crosslinked fabrics in combination with one and ten percent zeolite, and BIOgauze formulated with sodium zeolite and pectin. Table 6 summarizes some of the TEG clotting results of the ascorbic acid-crosslinked fabrics in combination with one and ten percent zeolite. However, this approach appears not to favor improved clotting profiles. On the other hand, as shown in Table 7, BIOgauze formulated with sodium zeolite and pectin demonstrated favorable clotting commensurate with hemorrhage control activity.

TABLE 6
Various Cotton Fabric swatches crosslinked with zeolite and Ascorbic acid

| | | % | R | | K | |
|---|---|---|---|---|---|---|
| Sample ID | Description | Add on | (min) | σ | (min) | σ |
| 062419-1 | TACGz CA + NaH₂PO₂ | 5.71 | | | | |
| 062419-2 | TACGz CA + SHP + Asc A | 4.93 | | | | |
| 062419-3 | TACGz CA + SHP + Asc A + 1% NaY | 6.93 | 8.8 | | 7.4 | |
| 062419-4 | TACGz CA + SHP + Asc A + 10% NaY | 8.86 | 8.7 | | 5.1 | |
| 062419-5 | B9S-2 CA + SHP | 5.50 | | | | |
| 062419-6 | B9S-2 CA + SHP + Asc A | 6.29 | | | | |
| 062419-7 | B9S-2 CA + SHP + Asc A + 1% NaY | 7.61 | | | | |
| 062419-8 | B9S-2 CA + SHP + Asc A + 10% NaY | 4.43 | 5.9 | 0.1 | 5.1 | 0.5 |
| 062419-9 | B8S-2 CA + SHP | 7.41 | | | | |
| 062419-10 | B8S-2 CA + SHP + Asc A | 6.74 | | | | |
| 062419-11 | B8S-2 CA + SHP + Asc A + 1% NaY | 6.97 | | | | |
| 062419-12 | B8S-2 CA + SHP + Asc A + 10% NaY | 3.75 | 5.8 | 0 | 6.4 | 0.7 |
| 062419-13 | Fine Mesh Gz CA + SHP | 4.79 | | | | |
| 062419-14 | FMGz CA + SHP + Asc A | 3.14 | | | | |
| 062419-15 | FMGz CA + SHP + Asc A + 1% NaY | 3.80 | | | | |
| 062419-16 | FMGz CA + SHP + Asc A + 10% NaY | 4.17 | 8.8 | 0.9 | 7.2 | 0.9 |
| 062419-17 | FMGz CA + SHP + Asc A | 5.81 | | | | |
| 062419-18 | FMGz, CA + SHP | 5.74 | | | | |
| 062019-11 | BIOGz CA + SHP | 4.01 | | | | |
| | Blood | — | 12.6 | 0.4 | 5.4 | 0.5 |
| | Procoagulant | — | 3.4 | | 2.5 | |

CA = citric acid, Asc A = Ascorbic acid, SHP = sodium hypophosphite monohydrate (NaH₂PCO₂•H₂O); TACGz = 60/20/20 greige cotton/polypropylene/bleach cotton blend hydroentangled nonwoven; B9S-2 = 100% greige cotton Hydroentangled nonwoven; B8S-2 = 100% bleached hydroentangled nonwoven; FMGz = 100% bleached fine mesh gauze(DeRoyal), BIOGz = previously treated TACGauze with ascorbic acid formulation.

TABLE 7
Pad-dry application of zeolite and pectin on BIOGauze

| | | % | R | | K | |
|---|---|---|---|---|---|---|
| Sample ID | Sample Description | Add-On | (min) | σ | (min) | σ |
| 050119-1 | 10% NaY only | 36.1 | 5.0 | 0.4 | 3.3 | 0.3 |
| 050119-2 | 0.25% Pectin only | 0.5 | 9.9 | 1.2 | 7.0 | 1.1 |
| 050119-3 | 0.5% Pectin only | 1.6 | 10.2 | 1.9 | 6.2 | 2.0 |
| 050119-4 | 0.25% PEC + 10% NaY | 27.4 | 5.4 | 0.7 | 3.7 | 1.4 |
| 050119-5 | 0.5% PEC + 10% NaY | 35.2 | 5.4 | 0.2 | 2.8 | 0.4 |
| | Blood | — | 13.6 | 0.9 | 7.6 | 1.4 |
| | Procoagulant | — | 4.3 | 0.3 | 3.3 | 0.8 |
| 062019-1 | 10% NaY only | 38.7 | 4.4 | 0.1 | 1.6 | 0.1 |
| 062019-3 | 1% CaCl₂ + 10% NaY | 53.5 | 4.6 | | 1.7 | |
| 062019-5 | 0.25% PEC + 10% NaY | 41.2 | 4.6 | 1.0 | 2.8 | 2.2 |
| 062019-6 | 0.25% PEC + 10% NaY | 36.8 | | | | |
| 062019-7 | 0.5% PEC + 10% NaY | 27.6 | 5.7 | 0.3 | 2.4 | 0.3 |
| 062019-9 | 0.5M Na₂CO₃ + 1% CaCl₂ + 10% | 37.0 | | | | |
| | NaY | | | | | |
| | Blood(bovine) | n/a | 19.6 | 2.2 | 8.7 | 1.7 |
| | Procoagulant | | 4.3 | 0.4 | 1.8 | 0.1 |

Abbreviations: Na₂CO₃ = sodium carbonate, PEC = pectin, NaY = sodium Y zeolite, CaCl₂ = calcium chloride

As shown in Table 8 the combination of sodium zeolite with pectin is somewhat comparable to employing alginate. When sodium carbonate and calcium chloride were employed in the formulation with pectin the time to clot formation was generally within the range expected for a procoagulant but time to fibrin formation was somewhat slower.

TABLE 8
Pad-dry* on BIOGauze

| | | % | R | | K | |
|---|---|---|---|---|---|---|
| Sample ID | Sample Description | Add-On | (min) | σ | (min) | σ |
| 050219-1 | 1% CaCl₂ + 10% NaY only | 44.5 | 4.4 | 0.5 | 2.0 | 0.1 |
| 050219-2 | 0.5% Na Alginate, 0.5M Na₂CO₃ + 1% CaCl₂ only | 16.4 | 12.3 | | 6.2 | |
| 050219-3 | 0.5% Na Alginate, 0.5M Na₂CO₃ + 1% CaCl₂ + | 28.9 | 6.9 | 0.4 | 2.7 | 1.4 |
| | 10% NaY | | | | | |
| 050219-4 | 0.5% Pectin, 0.5M Na₂CO₃ + 1% CaCl₂ only | 17.8 | 10.7 | | 3.5 | |
| 050219-5 | 0.5% Pectin, 0.5M Na₂CO₃ + 1% CaCl₂ + 10% NaY | 28.4 | 6.4 | 0.1 | 2.4 | 0.2 |
| 050219-6 | 0.5M Na₂CO₃, 1% CaCl₂, 10% NaY | 33.6 | 4.8 | 0.1 | 1.8 | 0.1 |
| 050219-7 | 0.5M NaOH, 1% CaCl₂, 10% NaY | 32.6 | 6.5 | 0.6 | 3.0 | 0.4 |
| 050219-8 | 10% NaY in 1 mM SDS | 17.8 | 6.2 | 0.4 | 2.0 | 0 |
| 050219-9 | 0.1M CaO, 1 mM SDS, 10% NaY | 34.5 | 5.8 | 0.1 | 2.0 | 0.3 |
| 050219-10 | 0.1M CaO, 1 mM SDS, only | 7.9 | 10.7 | 0.3 | 4.6 | 0.3 |
| | Blood | — | 12.7 | 2.2 | 4.9 | 0.4 |
| | Procoagulant | — | 3.3 | 0.0 | 1.4 | 0.1 |

*Stepwise reagent addition to padbath. Soak, pad then dry.
Abbreviations: Na₂CO₃ = sodium carbonate, CaO = Calcium oxide, SDS = Sodium dodecyl sulfate, Na Alginate = alginic acid, sodium salt

Table 9 shows that the use of calcium oxide did not improve on this trend. The use of spray applications to TACgauze showed comparable clotting times commensurate with procoagulant hemorrhage control. Abbreviations: TACGz=TACGauze, BIOGz=BIOGauze, PEC=pectin, NaY=sodium Y zeolite, CaCl2=calcium chloride, (S) spray.

TABLE 9
Comparison of spray to pad-dry method TEG data of TACGauze and BIOGauze*

| | | % | R | | K | |
|---|---|---|---|---|---|---|
| Sample ID | Sample Description | Add-On | (min) | σ | (min) | σ |
| | TACGz | | 9.7 | 0.8 | 5.3 | 0.7 |
| | | | 10.2 | | 6.5 | |
| | | | 9.6 | 0.8 | 4.8 | 1.0 |
| | BIOGZ | | 8.3 | 1.6 | 2.7 | 0.6 |
| | | | 10.6 | 0.4 | 8.7 | 3.0 |
| 082019-1 | TACGz 0.5% PEC, 2% CaCl2, | 40.4 | 5.2 | 0.0 | 1.4 | 0.1 |
| | 5% NaY | | 6.7 | 0.1 | 3.4 | 0.5 |
| | | | 4.9 | 0.1 | 1.5 | 0.2 |
| 022819-10 | TACGz 0.25% PEC, 10% NaY | 43.4 | 6.5 | 0.5 | 2.0 | 0.5 |
| | | | 6.6 | 0.6 | 3.4 | 0.1 |
| 050119-4 | BIOGz 0.25% PEC, 10% NaY | 27.4 | 5.4 | 0.2 | 1.0 | 0.1 |
| | | | 6.0 | 0.0 | 3.3 | 0.6 |
| 050219-1 | BIOGz 1% CaCl₂, 10% NaY | 44.5 | 5.1 | 0.0 | 1.4 | 0.2 |
| | | | 5.6 | 0.4 | 3.6 | 0.1 |
| | | | 4.2 | 0.1 | 1.2 | 0.3 |
| 081919-1 | TACGz 1% CaCl₂, 2% PEC, | 24.5 | 6.1 | | 1.3 | |
| | 10% NaY (S) | | 5.1 | 0.2 | 1.5 | 0.3 |
| 022819-5 | TACGz 0.5% PEC + 10% NaY | 45.7 | 6.1 | 1.0 | 1.5 | 0.1 |
| 050119-5 | BIOGz 0.5% PEC + 10% NaY | 35.2 | 5.8 | 0.2 | 1.4 | 0.1 |
| 031319-8 | TACGz 1% CaCl₂, 10% NaY | 28.8 | 5.1 | 0.2 | 1.5 | 0.5 |
| | bovine blood | | 12.9 | 0.4 | 2.9 | 0.2 |
| | | | 18.2 | 0.9 | 9.2 | 2.4 |
| | | | 13.4 | 0.2 | 4.0 | 0.4 |
| | Procoagulant | | 3.8 | 0.1 | 0.8 | 0.0 |
| | | | 3.8 | | 2.3 | |
| | | | 3.0 | 0.1 | 0.8 | 0.1 |

*In instances where the measurements were done during a different date/run, numbers for each run are on the same line.

We claim:

1. A fabric composition comprising attached a zeolite, or a zeolite/pectin complex.

2. The fabric composition of claim 1, wherein the fabric composition is a cloth, a woven fabric, a knitted fabric, a nonwoven fabric, or a final article.

3. The fabric composition of claim 2 wherein the fabric composition is a nonwoven fabric.

4. The fabric composition of claim 3, wherein the nonwoven fabric is a single layered fabric or a multilayered fabric.

5. The fabric composition of claim 4, wherein the nonwoven fabric is a single layered fabric comprising about 5% by weight to about 95% by weight non-scoured, non-bleached greige cotton fibers; about 5% by weight to about 95% by weight bleached cotton fibers; about 5% by weight to about 60% by weight hydrophobic fibers; all percentages adding up to 100 wt %.

6. The fabric composition of claim 5, wherein the nonwoven fabric comprises about 60% by weight non-scoured, non-bleached greige cotton fibers, about 20% by weight bleached cotton fibers, and about 20% by weight hydrophobic fibers.

7. The fabric composition of claim 2, wherein the fabric comprises about 85% by weight non-scoured, non-bleached greige cotton fibers, and about 15% by weight bleached cotton fibers.

8. The fabric composition of claim 4, wherein the nonwoven fabric is a multi-layered nonwoven fabric composition, comprising at least one inner layer containing about 50% by weight to about 95% by weight non-scoured, non-bleached greige cotton fibers and about 5% by weight to about 50% by weight hydrophobic fibers, all percentages adding up to 100 wt %, and at least one outer layer containing about 5% by weight to about 95% by weight non-scoured, non-bleached greige cotton fibers, about 5% by weight to about 95% by weight bleached cotton fibers, and about 5% by weight to about 60% by weight hydrophobic fibers, all percentages adding up to 100 wt %.

9. An article of manufacture prepared with the fabric composition of claim 1.

10. The article of manufacture of claim 9, wherein the article of manufacture is a medical textile.

11. The article of manufacture of claim 10, wherein the medical textile is a surgical arena fabric, a surgical personnel protective garment, a wound or non-wound patient dressing, a bandage, a gauze, a packing, or a cleaning material.

12. A method for preparing the fabric composition of claim 1, wherein the method comprises:
(a) saturating a fabric composition with treatment solution,
(b) padding the saturated fabric composition,
(c) drying the padded fabric composition at a first temperature, and
(d) curing the padded fabric composition at a second, higher temperature,
wherein the method optionally comprises, between step (c) and step (d), saturating the padded fabric composition with pectin, zeolite, pectin/zeolite complex or a mixture thereof, and drying the saturated padded fabric composition, or
wherein the method optionally comprises saturating the fabric composition with calcium chloride solution in step (a), followed by spraying pectin, zeolite, a pectin/zeolite complex, or a mixture thereof on both sides of the fabric composition, and drying the sprayed fabric composition.

13. The method of claim 12, wherein the method comprises:
(a) saturating a fabric composition with treatment solution,
(b) padding the saturated fabric composition,
(c) drying the padded fabric composition,
(d) saturating the padded fabric composition with pectin, zeolite, pectin/zeolite complex or a mixture thereof,
(e) drying the fabric composition containing pectin, zeolite, pectin/zeolite complex. or a mixture thereof at a first temperature, and
(f) curing the treated fabric composition at a second temperature that is higher than the first temperature.

14. The method of claim 12, wherein the method comprises:
(a) saturating a fabric composition with calcium chloride,
(b) padding the saturated fabric composition,
(c) spraying pectin, zeolite, a pectin/zeolite complex, or a mixture thereof on both sides of the padded fabric composition,
(d) drying the sprayed fabric composition at a first temperature, and
(e) curing the sprayed fabric composition at a second temperature that is higher than the first temperature.

15. The method of claim 12, wherein the fabric composition is a cloth, a woven fabric, a knitted fabric, a nonwoven fabric, or a final article.

16. The method of claim 15, wherein the fabric composition is a nonwoven fabric.

17. The method of claim 16, wherein the nonwoven fabric is a single layered fabric comprising about 5% by weight to about 95% by weight non-scoured, non-bleached greige cotton fibers;
about 5% by weight to about 95% by weight bleached cotton fibers; about 5% by weight to about 60% by weight hydrophobic fibers; all percentages adding up to 100 wt %.

18. The method of claim 16, wherein the nonwoven fabric comprises about 60% by weight non-scoured, non-bleached greige cotton fibers, about 20% by weight bleached cotton fibers, and about 20% by weight hydrophobic fibers.

19. The method of claim 12, wherein the fabric comprises about 85% by weight non-scoured, non-bleached greige cotton fibers, and about 15% by weight bleached cotton fibers.

20. The method of claim 16, wherein the nonwoven fabric is a multi-layered nonwoven fabric composition, comprising at least one inner layer containing about 50% by weight to about 95% by weight non-scoured, non-bleached greige cotton fibers and about 5% by weight to about 50% by weight hydrophobic fibers, all percentages adding up to 100 wt %, and at least one outer layer containing about 5% by weight to about 95% by weight non-scoured, non-bleached greige cotton fibers, about 5% by weight to about 95% by weight bleached cotton fibers, and about 5% by weight to about 60% by weight hydrophobic fibers, all percentages adding up to 100 wt %.
